(19) United States
(12) Patent Application Publication
(10) Pub. No.: US 2012/0283595 A1
(43) Pub. Date: Nov. 8, 2012

(54) CERVICAL TENACULUM AND METHODS OF USE

(75) Inventors: Stuart Curtis, Sandy, UT (US); Derek Bradley, Salt Lake City, UT (US); Creighton Petty, Salt Lake City, UT (US); Brent Call, Salt Lake City, UT (US); Erik Ostler, Bountiful, UT (US); Matthew Sorensen, Spanish Fork, UT (US)

(73) Assignee: BC OPS, LLC, Sandy, UT (US)

(21) Appl. No.: 13/461,645

(22) Filed: May 1, 2012

Related U.S. Application Data
(60) Provisional application No. 61/518,257, filed on May 2, 2011.

(51) Int. Cl. A61B 1/32; A61F 2/04; A61B 10/02; A61B 17/43

(52) U.S. Cl. 600/562; 600/210; 600/35; 600/37

(57) ABSTRACT

A cervical tenaculum device is disclosed that grasps the cervix in a manner to reduce trauma to the tissue, while providing unimpeded visual access and instrument access to the cervical os. The disclosed embodiments may include a bell configured to engage a vaginal portion of a cervix and create an annular suction chamber against a surface of the vaginal portion the cervix. The suction chamber encircles the external os and uniformly distributes negative pressure in the suction chamber around the engaged surface of the cervix. An opening through a middle portion of the bell allows access to the external os when the bell is engaged, such that the external os remains unobstructed. A method of grasping and manipulating the cervix to perform a gynecological procedure on a subject, using the embodiments of the disclosed cervical tenaculum device is also disclosed.

### CROSS-REFERENCE TO RELATED APPLICATIONS

The present application claims priority under 35 U.S.C. §119(e) to U.S. Provisional Application No. 61/518,257 filed on May 2, 2011, titled “CERVICAL TENACULUM”, the entire contents of which are hereby incorporated by reference.

### TECHNICAL FIELD

The present disclosure relates generally to medical devices and methods used during gynecological procedures. More specifically, the present disclosure relates to methods and apparatuses to grasp and/or manipulate the cervix.

### BRIEF DESCRIPTION OF THE DRAWINGS

The embodiments disclosed herein will become more fully apparent from the following description, taken in conjunction with the accompanying drawings. These drawings depict only certain typical embodiments that are non-limiting and non-exhaustive, and which will be described with additional specificity and detail through use of the accompanying drawings in which:

FIG. 1 is a perspective view of a cervical tenaculum, according to one embodiment.

FIG. 2A is a perspective view of a distal side of a cervical tenaculum, according to one embodiment.

FIG. 2B is a perspective view of a proximal side of the cervical tenaculum of FIG. 2A.

FIG. 3A is a cross-sectional view of the cervical tenaculum of FIGS. 2A and 2B prior to insertion through the vagina and adherence to the cervix.

FIG. 3B is a cross-sectional view of the cervical tenaculum of FIGS. 2A and 2B upon adherence to the cervix during a medical procedure.

### DETAILED DESCRIPTION

Many gynecological procedures require the use of a device which can grasp and manipulate the cervix so as to allow a clinician to access the cervical os. The cervix of a mammalian female is the lower, narrow portion of the uterus, where the uterus joins with the top end of the vagina. The cervix may have a cylindrical or conical shape and defines the cervical os.

The anatomy of a typical mammalian female reproductive tract is such that the cervix is at an angle relative to the vaginal canal. The angle can obstruct a clinician's view of the external os. Moreover, the angle may limit or even prevent medical instruments from being directly inserted into the cervical os, unless either the instruments are sufficiently flexible to bend with the angle between the vagina and the cervix, or traction is used to grasp the cervix and pull it into alignment with the vaginal canal. The latter method, use of traction, may presently be more commonly used to access the cervix and is chosen, at least in part, to allow the clinician to view the cervical os. Currently available devices that grasp and manipulate the cervix include a single or double tooth tenaculum. These devices grasp the cervix by piercing the tissue with sharp teeth. The piercings caused by the teeth can result in pain and bleeding. Lacerations can occur when the teeth and/or the tenaculum lose grip on the cervix. Furthermore, in procedures that require the cervix to be dilated, a large amount of force is placed on the tenaculum which can cause the cervix to tear because the force is placed over a small area. These lacerations can cause patient pain and discomfort and can prolong procedures due to required stitching, cauterization, and visual impairment from bleeding. Some estimate that 2.2% of dilation and curettage procedures result in cervical laceration/tearing.

Disclosed herein are devices and methods which grasp the cervix using uniformly distributed negative pressure over a greater area of tissue than currently available devices. Importantly, the device grasps the cervix without the use or aid of teeth or prongs that may pierce, and possibly tear, tissue during use. The disclosed devices and methods reduce pain associated with grasping the cervix with the currently available devices and methods and reduce or eliminate trauma caused by sharp teeth that pierce and tear tissue. The devices and methods disclosed herein also maintain an unobstructed field of view of the cervical os as well as instrument access into the uterus through the cervical os. In fact, a clinician may have a better field of view using the disclosed device due to the absence of bleeding from lacerations that currently available devices may cause. These and/or other advantages of various embodiments will be evident from the disclosure herein.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The term “adhered to” is given its normal definition as being held or maintained in direct physical contact.

The term “medical procedure” may be any procedure performed on the body of a subject to benefit said subject, either for therapeutic or cosmetic purposes.

The term “clinician” refers to any person performing a medical procedure, as defined herein, on a subject.

The term “gynecological” is given its normal definition as anything associated with the female reproductive organs and/or the female reproductive physiological systems.

The term “tenaculum” broadly includes devices and instruments for grasping and holding something.

It will be readily understood by one of skill in the art having the benefit of this disclosure that the components of the embodiments as generally described and illustrated in the figures herein could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale.

The disclosure provided herein in connection with any particular figure is analogously applicable to the disclosure provided in connection with other figures. Further, components described and labeled in one figure may be present in the embodiments of other figures whether or not the features are labeled or described in each instance.

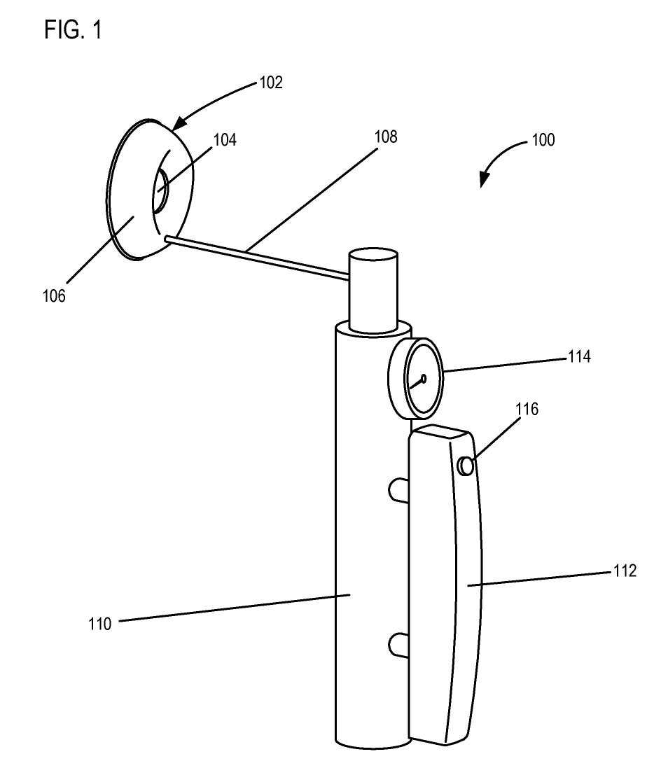

FIG. 1 is a perspective view of a cervical tenaculum 100, according to one embodiment. The cervical tenaculum 100 comprises a bell 102, a hollow rod 108, and a vacuum pump 110. The bell 102 is configured to engage a surface of a vaginal portion of a cervix and create an annular suction chamber against the surface of the vaginal portion the cervix. The bell 102 may completely encircle the external os and the annular suction chamber may completely encircle the external os to uniformly distribute negative pressure (e.g., a vacuum) created in the annular suction chamber over an entire annular region of the surface of the vaginal portion of the cervix. The vacuum created in the suction chamber against the surface of the cervix may entirely encircle the external os of the cervix.

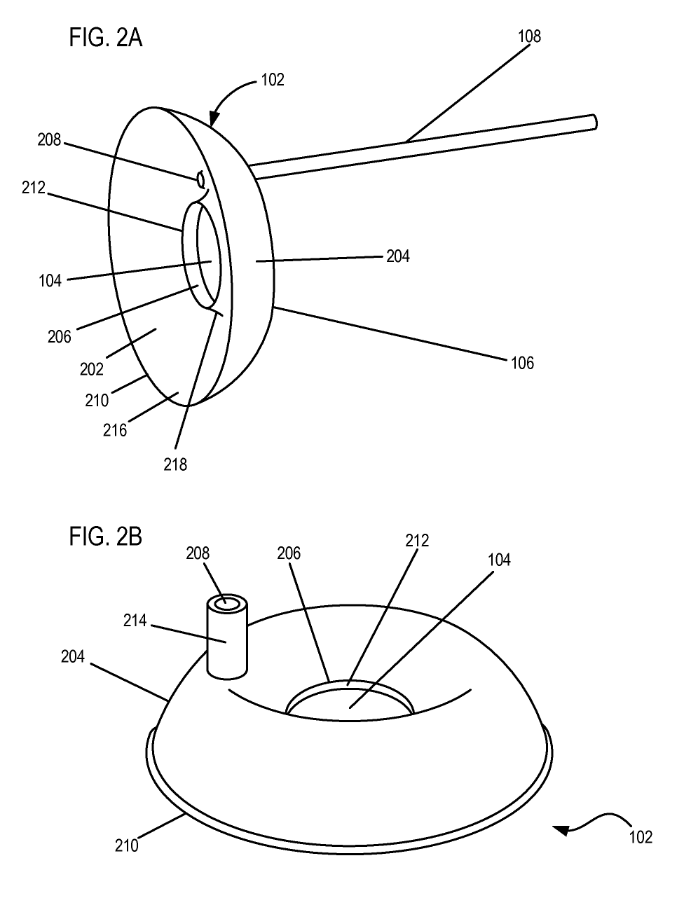

The bell 102 may be in the shape of a collapsed dome with an annular and convex upper surface 106 and an annular and concave under surface (shown in FIG. 2A) configured to conform to and/or engage a vaginal portion of a cervix. Described differently, the bell 102 may be generally in the shape of a hollow, radially bisected torus defining an annular, convex upper surface 106 and an annular, concave under surface (shown in FIG. 2A) configured to conform to and/or engage a vaginal portion of a cervix. The convex upper surface 106 faces a proximal direction relative to the clinician when the cervical tenaculum 100 is in use to grasp the cervix. The concave under surface faces in a distal direction (e.g., away from the clinician) when the device is in use to grasp the cervix.

The cervix of a mammalian female extends in a posterior inferior direction from the lower end of the uterus and joins with a posterior superior end of the vagina. In humans, the cervix has a cylindrical or conical shape and defines the cervical canal (or cervical os), which includes an internal os (or opening of the cervical canal into the uterine cavity) at an anterior superior end and an external os (or opening of the cervical canal into the vagina) at a posterior inferior end. A vaginal portion of the cervix protrudes through the upper anterior vaginal wall and defines the external os.

In a majority of human females, the vaginal portion of the cervix extends in a somewhat posterior or dorsal direction (or more specifically a posterior inferior direction) from the uterus, while the vagina extends in an anterior or ventral direction (or more specifically an anterior inferior direction) from the cervix. Accordingly, these two sections of the female reproductive tract form an angle that interferes with the clinician's view of the external os.

An annular apex of the vaginal portion of the cervix defines and surrounds the external os. The exterior (or extreme poterior inferior) portion of the vaginal portion of the cervix may define a lip and may have a generally torroidal or donut shape having a convex elliptical exterior surface that includes an outer surface or perimeter and an inner surface or perimeter. An extremity or most exterior edge of the lip of the vaginal portion may be referred to as the apex of the lip or apex of the vaginal portion.

The annular concave under surface (shown in FIG. 2A) of the bell 102 may include an outer surface and an inner surface and may be configured to conform to the lip and/or vaginal portion of the cervix by fitting over or around the lip of the vaginal portion of the cervix in a cupping configuration. An outer perimeter and/or distal edges of the under surface of the bell 102 may include an outer seal that be configured to form a seal around an outer circumference of the vaginal portion of the cervix. The outer seal may be configured to adhere to or engage the outer surface of the vaginal portion of the cervix at a region that is proximate (and posterior) to the fornix of the cervix and distal to an apex of the lip of the vaginal portion of the cervix.

An opening, or open center 104, may be disposed at a central region of the bell 102. The perimeter of the open center 104 may form or define an inner seal around an inner circumference of the vaginal portion of the cervix at a region that is within the apex of the vaginal portion of the cervix. In other words, the inner seal may be configured to adhere to or engage the inner surface of the lip of the vaginal portion of the cervix surrounding the external os, at a region that is more central or medial to the region adhered to by the outer seal. The inner seal may be disposed within the outer seal to form concentric elliptical seals surrounding the apex and/or lip of the vaginal portion of the cervix. The outer seal may be disposed distal to the inner seal during use of the cervical tenaculum 100. According to another embodiment, the inner seal may be configured to engage and adhere to the cervix at a region that is external to the apex of the vaginal portion of the cervix, on an outer surface, or outer portion of the lip, of the vaginal portion of the cervix.

In FIG. 1, the bell 102 is viewed from the proximal side, which is nearest to the vacuum pump 110. From this viewpoint, the convex upper surface 106 is visible. The open center 104 provides visual access and instrument access to the cervical os of the reproductive tract of the subject when the bell 102 is properly positioned during use. In this embodiment, a hollow rod 108 is connected to the bell 102 at the distal end of the hollow rod 108 and extends proximally toward a vacuum source, such as a hand-held vacuum pump 110, to which the hollow rod 108 is attached at its proximal end. The hollow rod 108 is rigid such that, when the bell 102 is adhered to and/or gripping the cervix of a subject, moving the hollow rod 108 in any direction results in the cervix being moved in the same direction. In this way, the clinician may manipulate the cervix to a desired position. Manipulation of the cervix, using the cervical tenaculum 100, may be performed by manually moving the vacuum pump 110, thus, indirectly maneuvering the hollow rod 108, which manipulates the bell 102 that is adhered to the cervix. The embodiment of FIG. 1 allows the clinician to manipulate the cervix with one hand while the other hand is free to, for example, perform a medical procedure.

In the embodiment in FIG. 1, the vacuum pump 110 may include a handle 112, a pressure gauge 114, and a pressure release valve 116. The handle 112 can be manipulated to create suction through the hollow tube 108. The handle 112 may be configured to fit to a single hand of a clinician such that the clinician may manipulate the vacuum pump 110 and cervical tenaculum 100 with one hand. The pressure gauge 114 allows the clinician to monitor the amount of negative pressure that is being applied to create a vacuum against the cervical tissue during use of the cervical tenaculum 100, so that an appropriate amount of negative pressure may be applied to hold the device firmly in place on the cervix, yet not damage the cervical tissue. The pressure release valve 116 may be manipulated by the clinician to release the negative pressure that secures the bell 102 to the cervix, thereby allowing disengagement of the bell 102 from the cervix. The handle 112 and pressure release valve 116 may enable a clinician to control and adjust the amount of negative pressure applied to the cervix. An amount of pressure can be maintained that is at least enough to securely adhere the bell 102 onto the cervix, thereby gripping the cervix to create enough traction to allow the clinician to manipulate the cervix, and less than may cause damage to the cervical tissue. The clinician may fully engage the pressure release valve 116 at the end of the medical procedure to release the bell 102, thereby allowing the device to be removed from the cervix and withdrawn through the subject's vagina.

When used properly, the clinician positions the cervical tenaculum 100 such that the concave under surface of the bell 102 is in direct contact with the vaginal portion of the cervix and such that the open center 104 is directly over the external os. The open center 104 allows a clinician performing a procedure to have an unobstructed field of view of the external os and instrument access to the cervical os and uterus. The vacuum pump 110 can be used to create negative pressure within a suction chamber or space between the cervical tissue and the concave under surface of the bell 102 and between the inner seal and the outer seal. The negative pressure creates enough traction to manipulate the cervix as needed to perform the medical procedure. Unlike currently available devices, the negative pressure is distributed evenly throughout and over a surface area of the cervix that is in contact with the concave under surface of the device. This configuration results in less pressure on any one area of tissue than may occur as a result of using currently available devices, which apply all the force needed to manipulate the cervix to one or two small areas where the sharp teeth pierce the tissue.

Once the device is adhered to the cervix, and the proper amount of negative pressure is applied, the clinician may apply force to the cervix in a variety of directions by manipulating an inflexible rod, such as the hollow rod 108, that extends from the bell 102. Depending on the embodiment at hand, the clinician may manipulate the inflexible rod either directly by grasping the inflexible rod or indirectly by grasping another component, such as the hand-held vacuum pump 110, to which the inflexible rod is coupled. In either embodiment, the inflexible rod allows the clinician to manipulate the cervix using only one hand, thereby leaving the other hand free to participate in the medical procedure.

Once the cervix is manipulated into the proper position, the clinician may insert instruments through the open center 104 to perform a procedure. The clinician has an unobstructed view of the cervical os to perform with greater precision medical procedures requiring access to the cervical os.

In an alternative embodiment, the inflexible rod (e.g., the hollow rod 108) may be connected to an automated system which moves the inflexible rod in a manner that does not require the clinician to manually manipulate the cervix through the inflexible rod. An example is a motorized device in which the clinician initiates movement of the inflexible rod through foot pedals that free both hands to perform the medical procedure. The inflexible rod may be hollow, as shown in the embodiment in FIG. 1, such that it not only provides leverage to manipulate the cervix, but also places the bell 102 in fluid connection with the vacuum source. Negative pressure produced by the vacuum source may thus be applied to the concave under surface through the inflexible, hollow rod 108.

In other embodiments, a vacuum source other than the hand vacuum pump 110 may be used to create negative pressure. The vacuum source may apply a negative pressure through a tubular member, such as the hollow rod 108. In one embodiment, the vacuum source may couple to the bell 102 of the cervical tenaculum 100 either through the hollow rod 108, as described above, or through a separate connection. In embodiment of FIG. 1, the hollow rod 108 may be attached to the bell 102, through one of a variety of attachment mechanisms, at a suction aperture 208 (shown in FIG. 2A) through the upper surface 106 of the bell 102. In another embodiment, the hollow rod 108 may be attached to the bell 102 through a combination of apertures and stabilization attachment sites for added strength and stability, wherein at least one aperture is in fluid communication with the vacuum source. The attachment mechanism may maintain an air-tight seal and provides a strong connection. In an alternative embodiment, flexible, hollow tubing may couple the device to the vacuum source, while a separate, inflexible rod separately performs the function of allowing the clinician to insert and align the bell with the cervix, either with one hand or using an automated system as disclosed above.

In another embodiment the vacuum source may be a central vacuum source which comprises a single vacuum pump that may be in fluid connection with multiple devices throughout the building. Tubing runs from the single vacuum pump to multiple rooms throughout the building, culminating at a connector in each room where a clinician may connect the device to the tubing, and thus, to the vacuum source. A central vacuum source may also comprise a pressure gauge at each point at which a clinician may access negative pressure, thus enabling the clinician to monitor the amount of negative pressure being provided by the vacuum source. The central vacuum source may also include a mechanism through which the clinician may adjust the amount of negative pressure that is optimal for the precise clinical situation.

As can be appreciated, creation of negative pressure may be accomplished in a variety of ways, including through use of the hand vacuum pump 110 shown in FIG. 1, use of an external vacuum machine, or use of another vacuum source in fluid communication with the suction chamber or space between the cervical tissue and the concave under surface of the bell 102 and between the inner seal and the outer seal.

The cervical tenaculum 100 may be used for any procedure which may involve manipulation of the cervix. Such procedures include, but are not limited to, dilation and curettage, fetal demise, abortion, in vitro fertilization, laparoscopic removal of hydatidiform mole, retained placenta removal, intrauterine device placement, intrauterine device removal, hysteroscopy, vaginal hysterectomy, endometrial biopsy, endometrial polypectomy, endometrial ablation, essure insertion, and cervical biopsy.

The bell 102 may be constructed from a somewhat rigid material, such as polyurethane or silicone, or any combination of plastics, rubber, and metal dipped in a coating of one of the aforementioned materials and sufficiently rigid to prevent the bell from collapsing while maintaining enough flexibility to conform to a cervix. The bell constructed from such material may be disposable, and thus designed for single use. The inflexible rod portion of the device may be constructed from autoclavable material, which allows for multiple uses.

A single embodiment of the cervical tenaculum 100 may include a bell 102 that is configured to fit over a variety of cervix sizes and shapes while maintaining the seal. This is because the effective use of the device does not require that the area between the inner and outer seals which grasps cervical tissue surrounding the cervical os be a precise distance from the os or precise surface area. Alternatively, the bell 102 may be manufactured in different sizes and shapes to suit the needs of the clinician and/or patient, which may vary due to parameters such as, but not limited to, patient anatomy, patient size, and the procedure to be performed with the device.

FIG. 2A is a perspective view of the bell 102 and hollow rod 108 of the cervical tenaculum 100 of FIG. 1, as viewed from generally the distal end and generally toward the concave under surface 202 that faces distal to the clinician during use. The perspective view of the bell 102 of FIG. 2 illustrates the convex upper surface 106 which may comprise an outer surface 204 and an inner surface 206. The inner surface 206 defines the open center 104. FIG. 2A also illustrates the concave under surface 202, which may be configured to conform to and/or adhere to or engage the cervix. The under surface 202 comprises an outer surface 216 and an inner surface 218.

The bell 102 of FIG. 2A is coupled to a hollow rod 108. The hollow rod 108 may be connected at its distal end to the bell 102 with a coupling mechanism that creates an air-tight connection, and that is strong enough to maintain the air-tight connection, while the hollow rod 108 is being manipulated by the clinician. The coupling mechanism is not visible in FIG. 2A because it is disposed on the upper surface 106 of the bell 102. The coupling mechanism may be disposed at and/or define a suction aperture 208. For example, the distal end of the hollow rod 108 may have external threads, which are configured to mate with internal threads on a connecter. The proximal end of the hollow rod 108 is configured to attach to a vacuum source. Connecting mechanisms such as those disclosed for use at the distal end of the hollow rod 108 may be used to connect the proximal end of the hollow rod 108 to the vacuum source.

Embodiments where the hollow rod 108 is detachable are disclosed, in part, because the hollow rod 108 may be constructed from an autoclavable material. Thus, the hollow rod 108 may be reusable with a disposable bell 102. Alternatively, the hollow rod 108 may be permanently attached to the bell 102, or the bell 102 and the hollow rod 108 may be manufactured as a single component. In such an embodiment, the hollow rod 108 may be made of material that is not autoclavable and the combined component may be a disposable, single-use product. Alternatively, both the bell 102 and hollow rod may be formed of an autoclavable material and can be sterilized for reuse.

The perimeter of the distal end of the outer surface 204 of the upper surface 106 and/or outer surface 216 of the under surface 202 of the bell 102 may define an outer seal 210. The outer seal may be configured to adhere to or engage the outer surface of the vaginal portion of the cervix at a region that is proximate to the fornix of the cervix and distal to an apex of the lip of the vaginal portion of the cervix. The perimeter of the open center 104 of the bell 102, at a distal end of the inner surface 206 of the upper surface 106 and/or inner surface 218 of the under surface 202 of the bell may define an inner seal 212. The inner seal may be configured to adhere to or engage the lip of the vaginal portion of the cervix at a region distal to an apex of the vaginal portion of the cervix on the inner surface of the lip surrounding the external os. The outer seal 210 and the inner seal 212 allow formation of an air-tight seal between the bell 102 and the cervical tissue.

In another embodiment, the outer seal 210 and the inner seal 212 may comprise a separately formed or integrally formed component distinct from the bell 102 and coupled to a distal edge of the bell 102. The outer seal 210 and the inner seal 212 may be constructed from the same material as the bell 102. Alternatively, the outer seal 210 and/or the inner seal 210 may be constructed from a material other than the material from which the bell 102 is formed. Such other material may be chosen to provide increased adherence to the tissue and/or to create a gentler interaction with delicate cervical tissue. Improved adherence of the inner seal 212 and outer seal 210 may be accomplished by performing a procedure to remove mucous or dry the cervix prior to applying the device.

When the bell 102 is in position against the cervix of a subject and a vacuum source is engaged, a negative pressure is created by air being pulled through the hollow rod 108 from the suction chamber or space defined by the inner seal 212, the outer seal 210, the under surface 202 of the bell 102, and the cervical tissue. A vacuum is created in the suction chamber against the cervix causing the bell 102, as specifically the outer seal 210 and the inner seal 212, to adhere to the cervix.

FIG. 2B is a perspective view of the bell 102 of FIG. 1 and FIG. 2A, as seen from generally the proximal side nearest the vacuum pump 110 of FIG. 1. A connector 214 of a coupling mechanism is shown uncoupled from the hollow rod 108 (FIGS. 1 and 2A). The connector 214 of the embodiment of FIG. 2B may be a collar coupled to the upper surface 106 of the bell 102 and configured to couple to the hollow rod 108. For example, the collar may include thread configured to mate with threads on the hollow rod 108.

In other embodiments, the connector 214 may include a connector collar having cut out portions or barbs designed to interact with components on a mated fitting that is coupled to the distal end of the hollow rod 108. When the hollow rod 108 is rotated, the barbs may come into contact with mating barbs and secure the connector 214 to the mating fitting on the hollow rod 108. In some embodiments one or more o-rings may be used in conjunction with the connector 214 to create an air-tight seal.

When the vacuum source is engaged, negative pressure is created underneath the bell 102 causing the bell 102 to adhere to the cervix. In the embodiment shown in FIG. 2B, the “collapsed” depression at a middle portion of the upper surface 106 of the bell 102 may be fairly acute (e.g., deep). In other embodiments, the depression may be more shallow. In general, the relative proportions of the bell 102 may vary with different embodiments. Such embodiments may be designed for general use in gynecological procedures or optimized for specific procedures and/or instruments.

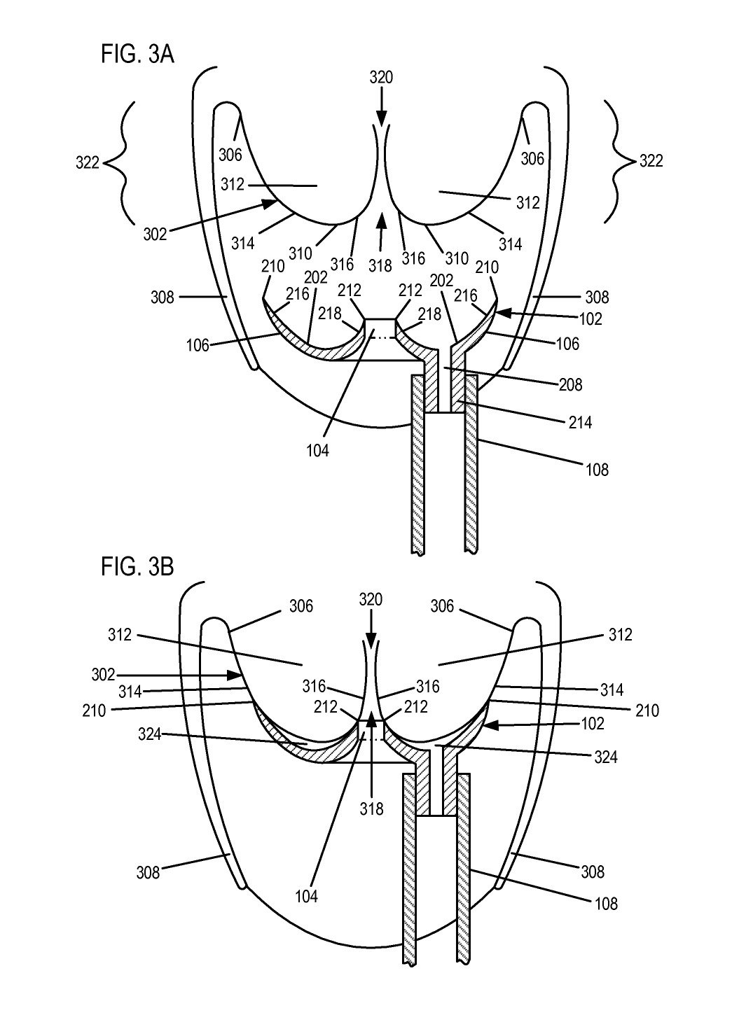

FIG. 3A is a cross-sectional view of the bell 102 and hollow rod 108 of FIG. 2A prior to adherence to the cervix 302 of a subject. The drawing shows a cross-sectional view of the vaginal wall 308 and cervix 302 to illustrate positioning of the bell 102 as the clinician inserts the bell 102 into the vagina, with the concave under surface 202 facing toward the cervix 302.

The cervix 302 is shown with the apex 310 pointing downward in FIG. 3A. The cervix 302 defines the cervical canal 320 (or cervical os), which includes an internal os (not shown) at an anterior end and an external os 318 (or opening of the cervical canal into the vagina) at a posterior end. A vaginal portion 322 of the cervix 302 (also termed the ectocervix) protrudes through the upper vaginal wall 308 and defines the external os 318. The exterior (or extreme) portion of the vaginal portion of the cervix may define an annular lip 312 around the external os 318 and may have a generally torroidal or donut shape that defines an outer surface 314 (or outer perimeter) and an inner surface 316 (or inner perimeter). An extremity or most exterior edge of the lip 312 of the vaginal portion 322 may be referred to as the apex 310 of the lip or apex 310 of the vaginal portion 322.

The bell 102 is inserted into the vagina and oriented to engage or fit over the cylindrical cervix 302 in a cupping (or “cup-like”) manner. The bell 102 is positioned such that the open center 104 is proximal to and encircling the external os 318 of the cervix 302. The under surface 202 is facing the cervix 302. The outer seal 210 is positioned to engage the outer surface 314 of the lip 310 of the vaginal portion 322 of the cervix 302. The inner seal 212 is positioned to engage the inner surface 316 of the lip 312 of the vaginal portion 322 of the cervix 302.

FIG. 3B is a cross-sectional view of the bell 102 of FIG. 3A properly positioned for use during a medical procedure. The outer seal 210 is positioned proximate to the fornix 306 and distal to the external os 318. The inner seal 212 is positioned proximal to the outer seal 210 and on the inner surface 316. The two seals 210, 212, thus, form concentric ellipses surrounding the external os 318 with the inner seal 212 located nearer the external os 318 relative to the outer seal 210.

With the inner seal 212 in contact with the inner surface 316 of the vaginal portion 322 of the cervix 302 and the outer seal 210 in contact with the outer surface 314 of the vaginal portion 322 of the cervix 302, a suction chamber 324 is created. Suction applied through the suction aperture 208 and hollow rod 108 creates a vacuum in the suction chamber 324, which causes the bell 102, and specifically the inner seal 212 and the outer seal 210, to adhere to and create a grip on the vaginal portion 322 of the cervix 302.

The open center 104 encircles the external os 318 to provide the clinician both with access into the external os 318 and, when the cervix 302 is properly maneuvered by manipulating the hollow rod 108, a clear view of the external os 318. The hollow rod 108 is connected to, and in fluid communication with, the bell 102 through a connector 214. The hollow rod 108, in turn, is connected to the vacuum source (not shown). Once the vacuum source is engaged, the negative pressure under the bell 102 causes the bell 102, and specifically the outer seal 210 and the inner seal 212 to adhere to the cervix 302. A vacuum is created in the suction chamber 324 or space between the cervix 302 and the concave under surface 202 of the bell 102 and between the inner seal 212 and the outer seal 210. The vacuum creates traction on the cervix 302. The traction created keeps the bell 102 secure so that the clinician may manipulate the cervix 302 to a position that is ideal for achieving an optimal view of the cervix 302 and for performing a medical procedure by maneuvering the hollow rod 108.

In another embodiment, the inner seal 212 may be positioned to engage and adhere to an outer surface 314 of the vaginal portion of the cervix 302. As can be appreciated, in other embodiments the inner seal 212 and/or the outer seal 210 may be configured to engage and adhere to the vaginal portion 322 of the cervix 302 at any position along the lip 312 of the vaginal portion of the cervix 302, ranging from inside the apex 310 of the lip 312 (as illustrated in FIG. 3B), to the apex 310 of the vaginal portion of the cervix 302, and to a region of the outer surface 314 of the cervix 302 distal to the apex 310.

While specific embodiments of the cervical tenaculum and methods for use in connection with the cervical tenaculum have been illustrated and described, it is to be understood that the disclosure provided is not limited to the precise configuration and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It should be appreciated that in the above description of embodiments, various features are sometimes grouped together in a single embodiment, figure, or description thereof for the purpose of streamlining the disclosure. It will be apparent to those having skill in the art that changes may be made to the details of the above-described embodiments without departing from the underlying principles of the disclosure herein.

We claim:

1. A cervical tenaculum comprising:
a bell configured to engage a vaginal portion of a cervix and create an annular suction chamber against a surface of the vaginal portion the cervix, the annular suction chamber encircling an external os of the cervix and uniformly distributing negative pressure created in the suction chamber to create a vacuum against an annular region of the surface of the cervix around the external os of the cervix, the bell defining an opening through a middle portion of the bell and configured to encircle and allow access to an external os of the cervix as the bell is engaged with the cervix, such that the external os remains unobstructed as the bell is engaged with the cervix;
a rod coupled to the bell to position the bell in engagement with the vaginal portion of the cervix, the rod secured to the bell to cause manipulation of the bell as the rod is manipulated and to enable manipulation of the cervix as the vacuum in the suction chamber adheres the bell to the cervix; and
a vacuum source in fluid communication with the suction chamber created as the bell engages the surface of the vaginal portion of the cervix, the vacuum source configured to, upon activation, apply negative pressure to the suction chamber to create a vacuum against the surface of the vaginal portion of the cervix to adhere the bell to the cervix.

2. The cervical tenaculum of claim 1, wherein the bell comprises:
an annular concave under surface forming an inner surface and an outer surface, the under surface configured to engage the surface of the vaginal portion of the cervix and thereby form the annular suction chamber; and
an upper surface forming an inner surface and an outer surface, the inner surface defining the opening through the middle portion of the bell, the opening extending to the under surface of the bell.

3. The cervical tenaculum of claim 2, wherein the bell further comprises:
an outer seal disposed at a distal edge of the outer surface of the under surface, the outer seal configured to engage an annular region of the surface of the vaginal portion of the cervix; and
an inner seal disposed at a distal edge of the inner surface of the under surface, the inner seal configured to engage an annular region of the surface of the vaginal portion of the cervix within the annular region engaged by the outer seal,
wherein the under surface, the outer seal, and the inner seal define the annular suction chamber between the surface of the cervix and the under surface and between the outer seal and the inner seal as the bell is engaged with the cervix.

4. The cervical tenaculum of claim 3, wherein the outer seal of the bell is configured to engage an annular region of the surface of the cervix proximate to a cervical fornix and distal to and outside of an apex of the vaginal portion of the cervix.

5. The cervical tenaculum of claim 3, wherein the inner seal of the bell is configured to engage an annular region of the surface of the cervix on an inner surface of the cervix, within an apex of the vaginal portion of the cervix.

6. The cervical tenaculum of claim 2, wherein the bell further comprises:
a suction aperture defined in the upper surface of the bell and in fluid communication with the suction chamber to transfer a suction generated by the vacuum source to the suction chamber and thereby create negative pressure in the suction chamber and create a vacuum against the surface of the vaginal portion of the cervix.

7. The cervical tenaculum of claim 1, further comprising:
a tubular member configured to couple the bell to the vacuum source and to transfer a suction generated by the vacuum source to the suction chamber.

8. The cervical tenaculum of claim 7, wherein the tubular member is a lumen through the rod.

9. The cervical tenaculum of claim 7, wherein the tubular member comprises a hollow rod.

10. The cervical tenaculum of claim 7, wherein the tubular member comprises a section of flexible tubing.

11. The cervical tenaculum of claim 7, wherein at least one of the tubular member and bell is autoclavable.

12. The cervical tenaculum of claim 1, wherein the vacuum source is a vacuum pump.

13. The cervical tenaculum of claim 12, wherein the vacuum pump is a hand vacuum pump.

14. The cervical tenaculum of claim 1, wherein the vacuum source is a central vacuum source.

15. The cervical tenaculum of claim 1, wherein the vacuum source comprises a pressure gauge.

16. A method for grasping and manipulating the cervix to perform a gynecological procedure on a subject, the method comprising:
inserting a bell of a cervical tenaculum into a vagina of the subject using a rod coupled to the bell, the bell configured to engage a surface of a vaginal portion of a cervix and create an annular suction chamber against the surface of the vaginal portion the cervix, the annular suction chamber encircling an external os of the cervix, the bell defining an opening through a middle portion of the bell and configured to encircle and allow access to an external os of the cervix as the bell is engaged with the cervix, such that the external cervical os remains unobstructed as the bell is engaged with the cervix;
manipulating the rod to position the bell in engagement with the surface of the vaginal portion of the cervix and create the annular suction chamber against the surface of the vaginal portion of the cervix;
activating a vacuum source in fluid communication with the suction chamber to generate and uniformly distribute a negative pressure created in the suction chamber to create a vacuum against an annular region of the surface of the cervix around the external os of the cervix and thereby adhere the bell to the cervix and cause the bell to grip the cervix;
manipulating the rod coupled to the bell of the cervical tenaculum to manipulate the bell as it grips the cervix and thereby manipulate the cervix to a desired position to perform the gynecological procedure.

17. The method of claim 16, further comprising:
positioning the opening through the bell over and encircling an external os of the cervix of the subject, such that the external os is visible and accessible through the open center.

18. The method of claim 16, further comprising:
performing a desired gynecological procedure, including inserting instruments through the opening through the bell and into the external os of the cervix of the subject.

19. The method of claim 18, wherein the gynecological procedure comprises one of dilation and curettage, fetal demise, abortion, in vitro fertilization, laparoscopic removal of hydatidiform mole, retained placenta removal, intrauterine device placement, intrauterine device removal, hysteroscopy, vaginal hysterectomy, endometrial biopsy, endometrial polypectomy, endometrial ablation, essure insertion, and cervical biopsy.

20. The method of claim 16, wherein the bell of the cervical tenaculum comprises:
an annular concave under surface forming an inner surface and an outer surface, the under surface configured to engage the surface of the vaginal portion of the cervix and form the annular suction chamber
an upper surface forming an inner surface and an outer surface, the inner surface defining the opening through the middle portion of the bell, the opening extending to the under surface of the bell;
an outer seal disposed at a distal edge of the outer surface of the under surface, the outer seal configured to engage an annular region of the surface of the vaginal portion of the cervix;
an inner seal disposed at a distal edge of the inner surface of the under surface, the inner seal configured to engage an annular region of the surface of the vaginal portion of the cervix within the annular region engaged by the outer seal,
wherein the under surface, the outer seal, and the inner seal define the annular suction chamber between the surface of the cervix and the under surface and between the outer seal and the inner seal as the bell is engaged with the cervix.

21. A bell of a cervical tenaculum to grip a cervix, the bell comprising:
an annular concave under surface forming an inner surface concentric with an outer surface, the under surface configured to engage the surface of the vaginal portion of the cervix and thereby form the annular suction chamber to fully encircle an external os of the cervix and uniformly distribute negative pressure generated in the suction chamber to create a vacuum against an annular region of the surface of the cervix around the external os of the cervix; and
an upper surface forming an inner surface and an outer surface, the inner surface defining an opening through a middle portion of the bell, the opening extending to the under surface of the bell.

22. The bell of a cervical tenaculum of claim 21, the further comprising:
an outer seal disposed at a distal edge of the outer surface of the under surface, the outer seal configured to engage an annular region of the surface of the vaginal portion of the cervix outside an apex of the vaginal portion of the cervix; and
an inner seal disposed at a distal edge of the inner surface of the under surface and concentric to the outer seal, the inner seal configured to engage an annular region of the surface of the vaginal portion of the cervix within the annular region engaged by the outer seal, wherein the under surface, the outer seal, and the inner seal define an annular suction chamber between the surface of the cervix and the under surface and between the outer seal and the inner seal as the bell is engaged with the cervix, and wherein a suction transferred within the suction chamber, as the bell is engaged with the cervix, creates a vacuum in the suction chamber and against the surface of the vaginal portion of the cervix to adhere the bell to the cervix.

23. The bell of a cervical tenaculum of claim 21, further comprising:
a suction aperture in the upper surface and in fluid communication with the annular suction chamber that is formed between the surface of the cervix and the under surface and between the outer seal and the inner seal as the bell is engaged with the cervix, the suction aperture configured to transfer a suction of a vacuum source to the suction chamber as the vacuum source is activated.
